(19) United States
(12) Patent Application Publication
(10) Pub. No.: US 2016/0256255 A9
(43) Pub. Date: Sep. 8, 2016

(54) DESIGN AND METHODS FOR A DEVICE WITH BLOOD FLOW RESTRICTION FEATURE FOR EMBOLUS REMOVAL IN HUMAN VASCULATURE

(71) Applicant: Jianlu Ma, Irvine, CA (US)

(72) Inventor: Jianlu Ma, Irvine, CA (US)

(21) Appl. No.: 14/082,019

(22) Filed: Nov. 15, 2013

Related U.S. Application Data
(60) Provisional application No. 61/832,786, filed on Jun. 8, 2013.
(60) Provisional application No. 61/768,336, filed on Feb. 22, 2013.

(51) Int. Cl. A61F 2/01

(52) U.S. Cl. CPC A61F 2/01

(57) ABSTRACT

A mechanical thrombectomy device system is disclosed that is made from a single piece of biocompatible material, including a proximal flow block portion/feature, and/or, a flow block feature in the device body portion, a guidewire like delivery portion and an expandable, treatment portion. The construction of the device from a single piece allows for a seamless transition from the delivery portion to the treatment portion, thus removing any joints or bonding of the two portions together as separate pieces. This improves the strength of the system as a whole and greatly reduces the possibility of the two parts unintentionally detaching from each other. Likewise, the distal treatment portion is cut from a piece of material the same size as the proximal delivery portion, allowing the device to be compacted to a similar size profile giving it delivery advantages including a lower delivery force required and requiring small access systems, and the treatment portion's surface can be altered to enhance embolus affinity by either coating with a substance or changing the texture by mechanical or chemical means.

### CROSS-REFERENCE TO RELATED APPLICATIONS

This application claims the full Paris Convention benefit of, and priority to, U.S. Provisional Application Ser. No. 61/768,336, filed on Feb. 22, 2013, and U.S. Provisional Application Ser. No. 61/832,768, filed on Jun. 7, 2013, the contents of each of which are incorporated by this reference as if fully set forth herein in their entirety.

### FIELD OF THE DISCLOSURE

This invention generally relates to devices and methods useful for emboli retrieval and removal devices to treat, among other things, ischemic stroke. In particular, this invention relates to a medical device that can be used as a mechanical thrombectomy device to retrieve and remove an obstruction responsible for a narrowing and/or blockage of the vessel(s) in neurovasculature or cardiac vasculature to restore oxygenated blood flow or superoxygenated blood distal of the blockage after the obstruction is being cleared.

### BACKGROUND OF THE DISCLOSURE

This invention relates to medical mechanical thrombectomy devices and more particularly to collapsible and expandable devices and methods for increasing blood flow through an obstructed blood vessel in neurovasculature and/or cardiac vasculature. This device can also be used to treat obstructed vessels in peripheral vasculature, such as in Deep Vein Thrombosis and related conditions, symptoms and disease states.

Currently, the FDA-approved treatment options for an acute ischemic stroke include intravenous (IV) delivery of clot dissolving medicine; and mechanical thrombectomy devices.

For treatment use clot dissolving medicine, the thrombolytic agent (Tissue Plasminogen Activator (t-PA)) is injected into the vasculature to dissolve blood clots that are blocking blood flow to the neurovasculature. Intravenous t-PA is currently limited in use because it must be used within a three hour window from the onset of a stroke and can result in an increased risk of bleeding. This standard of care leaves room for upgrading, lower aisle profiles and is only the appropriate approach to treatment for a limited class of individuals, groups and temporally-limited exigent cases.

The second option includes using mechanical thrombectomy devices. Such devices are designed to physically capture an embolus or clot and remove it from the blocked vessel, thereby restoring blood flow. The major advantage of the mechanical thrombectomy device is it can expand the treatment windows from 3 hours to over 10 hours.

Some existing mechanical thrombectomy devices used for increasing blood flow through an obstructed blood vessel include: 1) a filter trap designed and built to collect and remove emboli; 2) a cork-screwed guidewire like device to retrieve embolus; 3) a stent like device connected to a delivery wire to retrieve embolus. The major disadvantages of above mentioned existing mechanical thrombectomy devices include: 1) for filter like devices, filters tend to be cumbersome and difficult to delivery, deploy and a larger profile guide catheter may be needed to fully remove the embolus. In addition, it is difficult to coordinate precisely and predictably a desired movement to position the device properly in the vessel. The device can drift within the vessel, twist, or not be adequately conforming to the vessel wall and, therefore not effective for removing embolus; 2) for cork-screwed guidewire-like device, often they can only capture and remove embolus that is firm or is subject to certain mechanical variables such as being held together by itself as one piece.

There is no immediate vascular recanalization during the procedure and the device is not capable of capturing small emboli that break off from the large embolus if any; 3) the existing stent like mechanical thrombectomy device is not capable of capturing small emboli that break off from the large embolus if any, and can lead to complications such as blockage of distal smaller vessels, vessel dissection, perforation and hemorrhage arise as a result of over-manipulation in the vessel.

A common disadvantage from the above mentioned existing devices include 1) the device may capture an embolus, but then lose grasp of it and migrate/deposit it incidentally in another area of the neurovasculature, creating the potential for a new stroke in a different part of the neurovasculature; 2) the device is not capable to capture the small embolus break off from the major embolus and prevent it from migrating to a more distal area of the neurovasculature; 3) the relative large device profile prevents it from treating the distal small diameter vessels.

Another disadvantage to existing mechanical thrombectomy devices is that they are built using two or more distinct pieces that require either joints or bonding between the delivery system and the treatment device. This connection of the pieces generally results in a weakness in the device that can result in an unintentional separation of the two pieces, possibly leaving the treatment device in the body during embolus retrieval. Also, the treatment portion of mechanical thrombectomy devices (particularly stent like devices) tend to be cut from tubing that is larger than the delivery system, thus making the treatment portion the limiting factor in terms of minimizing the compacted profile of the device, requiring larger access systems and greater delivery force to deliver the device.

Other flaws in the current mechanical thrombectomy designs include poor visibility/radiopacity, lack of variation in the delivery portion to enhance and improve deliverability, and lack of coatings or modified surface textures on the treatment portion to enhance embolus affinity, etc. In conclusion, there is a great need for improved devices, device systems, and methods for increasing blood flow through a blood vessel as described herein. None of the existing medical mechanical thrombectomy devices address all necessary needs to date.

### SUMMARY OF THE DISCLOSURES

Briefly stated, a mechanical thrombectomy device system is disclosed that is made from a single piece of biocompatible material, including a proximal flow block portion/feature, and/or, a flow block feature in the device body portion, a guidewire like delivery portion and an expandable, treatment portion. The construction of the device from a single piece allows for a seamless transition from the delivery portion to the treatment portion, thus removing any joints or bonding of the two portions together as separate pieces. This improves the strength of the system as a whole and greatly reduces the possibility of the two parts unintentionally detaching from each other. Likewise, the distal treatment portion is cut from a piece of material the same size as the proximal delivery portion, allowing the device to be compacted to a similar size profile giving it delivery advantages including a lower delivery force required and requiring small access systems, and the treatment portion's surface can be altered to enhance embolus affinity by either coating with a substance or changing the texture by mechanical or chemical means.

A medical mechanical thrombectomy device and methods useful for increasing blood flow through a blood vessel are described herein. In general, a device system includes an elongate member (proximal portion) and an expandable member (distal portion) fabricated from a single piece of super elastic or shape memory biocompatible material (tubing). The expandable member is configured to be inserted into a blood vessel and defines multiple spaces/openings in a wall of the expandable member. The expandable member generally has a compacted configuration for delivery and insertion into the target location of a blood vessel and an expanded configuration in which the expandable member to engage/receive embolus/clots with the multiple space/openings on it. The proximal portion/end of the expandable member has a flow block feature to block the blood flow when the device is expanded during the procedure.

The expandable member includes a first component having a stent like structure with multiple space/openings in its wall to help engage the embolus/clot and establish structural integrity of the device.

The profile of the treatment portion is not “smooth”. It contains “peaks” and “valleys” formed by the spaces/openings along the length. The major frame of the “peaks” and “valley” is formed by two or more “spines” in helix/spiral configuration. The “peaks”, “valleys”, and spiral “spines” help to improve the embolus affinity for better clot adhesion during procedure. The blood flow block feature can also be built into the device body (working length) area to block the flow during procedure. One example is to cover the “Valley” area in the device body, so that the blood flow cannot go through the device/vessel lumen when the device is expanded, which helps the device to engage the clot and prevent/reduce the clots break a part or being flush away to the distal vasculature

The strut(s) in the stent like structure forms angles with the longitudinal axis of the device in the range from at least about 5 to approximately 175 degrees. The strut(s) can have twists along their longitudinal axes.

The treatment portion has a tapered distal section collecting small embolus break offs from major clot(s) and preventing them from migrating to a more distal area of the neurovasculature.

The device treatment portion can have flow block feature at its proximal portion to block the blood flow when device is expanded during the procedure. FIGS. 5 and 7 each shows some exemplary configurations of the proximal flow block feature.

The device body can have flow block feature along the length. FIG. 6 shows some exemplary configurations of the flow block feature in the expandable portion of the device.

The device can be made from either metallic biocompatible material (such as Nitinol, stainless steel, Co—Cr base alloy, Ta, Ti, etc.) or polymer based biocompatible material (polymers with shape memory effect, PTFE, HDPE, LDPE, Dacron, Polyester, etc.). For ischemic stroke treatment, the expandable stent-like member must be flexible enough to negotiate the torturous vasculature of the brain and without modifying the vessel profile at the target location. The profile of the expandable stent-like member must be small enough to reach target treatment site as known to artisans.

The expandable member can be fully or partially coated with chemical(s), drug(s) or other bioagents to prevent clotting and/or for the better adhesion between the device and embolus. The device surface can be treated to form different surface layer (oxidation layer, Nitro or carbonized or N—C-combined surface layer, etc.) for better adhesion between device and embolus. The device strut surface can be mechanically, chemically, or electrochemically treated to form “rough” surfaces for better adhesion between devices and emboli.

Radiopaque markers (marker coils, marker bands, Radiopaque wire(s), Radiopaque coatings, etc.) are integrated into the treatment device on the distal portion and proximal portion; or through the entire inner lumen of the treatment portion either partially or entirely to help position the device under standard fluoroscopy equipment.

The transition portion of the device, where the proximal and distal portions meet will be seamless requiring no joints or bonding. Also, the transition portion can be modified with a number of variations to vary flexibility by having straight tubing, spiral cut through the wall thickness, or spiral cut partially through the wall thickness. When spiral cut, the flexibility can be varied through variable pitch sizes across the length. The transition portion can be covered by polymer tubing/layers/covers for the optimization of the device deliverability and the surface smoothness.

The inner lumen in the entire device can be used for the local drug delivery in the vasculature if needed. Following paragraphs describe the details of each device component design.

### BRIEF DESCRIPTION OF THE DRAWINGS

For a more complete understanding of the invention, reference is hereby made to the drawings, in which:

FIG. 1 is an example of the overall profile of the device, according to embodiments of the present disclosure;

FIG. 2 is an example of the distal portion (treatment portion) of the devices.

FIG. 3a is an example of the transition portion of the device with radiopaque material inserted into the lumen of the tubing and having larger dimensions at both ends (dumbbell shape), or can be attached onto the geometry.

FIG. 3b is an example of a transition portion of the device with a spiral cut through the entire wall thickness.

FIG. 3c is an example of a transition portion of the device with a spiral cut partially through the entire wall thickness.

FIG. 4a is an example of a transition portion of the device with a spiral cut configuration showing variable pitch sizes.

FIG. 4b is an example of a transition portion of the device with a spiral cut configuration through the entire wall thickness.

FIG. 5 is an exemplary configuration of a proximal flow block feature/element on the proximal portion of the expandable portion.

FIG. 6 is an exemplary configuration of a flow block feature/element on the body portion of the expandable portion.

FIG. 7 is an exemplary configuration of the proximal flow block feature/element on the proximal portion of the expandable portion.

### DETAILED DESCRIPTION

The present inventor has discovered myriad benefits associated with having blood flow restriction features incorporated within uniquitous systems, devices and apparatus.

Briefly stated, a mechanical thrombectomy device system is disclosed that is made from a single piece of biocompatible material, including a proximal flow block portion/feature, and/or, a flow block feature in the device body portion, a guidewire like delivery portion and an expandable, treatment portion. The construction of the device from a single piece allows for a seamless transition from the delivery portion to the treatment portion, thus removing any joints or bonding of the two portions together as separate pieces. This improves the strength of the system as a whole and greatly reduces the possibility of the two parts unintentionally detaching from each other. Also, because the distal treatment portion is cut from a piece of material the same size as the proximal delivery portion, it allows the device to be compacted to a similar size profile giving it delivery advantages including a lower delivery force required and requiring small access systems. Additional delivery advantages from this design include the ability to manipulate the flexibility of the delivery system by varying the pitch size. In addition, a radiopaque marker can be attached within the lumen of the device to improve visualization. Lastly, the treatment portion's surface can be altered to enhance embolus affinity by either coating with a substance or changing the texture by mechanical or chemical means.

Compared with existing mechanical thrombectomy devices, the unique device design included in this invention has the advantage of 1) having proximal flow block/restriction feature to block the blood distal flow when the device is deployed during use; this feature can help to eliminate or reduce the risk of flush or break the clots during the procedure; 2) being made from a single piece of Nitinol super elastic material (such as tubing, etc.), Nitinol shape memory alloy material, or other biocompatible materials which exhibit super elastic or shape memory properties, thus giving the device a seamless transition from proximal delivery portion to distal therapeutic portion. This effectively removes any joints or bonding of a delivery wire with the treatment device, eliminating this physical weakness in the device and greatly reducing unintentional breakages during device delivery/retrieval. Another important advantage of the design disclosed in present invention is varies features (such as spiral cut, helix/coil configuration, etc.) can be implemented into device proximal delivery portion to achieve variable flexibility for easy delivery and navigation. The flexibility of the proximal delivery portion can vary from proximal to distal. For example, the distal portion can be more flexible than proximal portion. Furthermore, the device can achieve a smaller compacted profile, which reduces delivery and retrieval force and allows the physician to use smaller microcatheters for delivery to smaller vessels or the more distal vasculature. During procedure, this proximal block/restriction portion/feature can block the blood flow through the lumen of the device and the lumen of the treatment vessel segment, to help engage the clot and eliminate or reduce the risk to break the clot or flush the clots distal to the more distal vasculature.

Although detailed descriptions of the invention are disclosed herein, it needs to be understood that the disclosed descriptions are merely exemplary of the invention that may be embodied in various and alternative forms based on the basic idea or design principal disclosed. Specific structural and functional details disclosed herein are not to be interpreted as limiting, but merely as a basis for teaching skilled ones in the art to variously employ the vasculature mechanical thrombectomy device embodiments.

What is essential is that the device described in the present invention overcomes the shortcomings of the existing technologies and can be delivered to the target vasculature smoothly, retrieved safely, and remove the entire embolus. In use, the mechanical thrombectomy device described in the present invention can be compacted to a low profile and loaded onto a delivery system and delivered to the target location in the vessel by a medical procedure such as by use of a delivery catheter. The mechanical thrombectomy device can be released from the delivery system when it reaches the target implant site and recover to its normal expanded profile by the elastic energy stored in the device (self-expandable device).

As for the relative position of the device in relation to the embolus, it can either be deployed at the site of the embolus, or deployed distal to the embolus. In dealing with long embolus, the device can also be used to remove the embolus from the proximal portion to distal with multiple passes, until entire embolus is removed. The present invention offers the advantage of having a seamless transition from delivery portion to treatment portion from being fabricated from a single piece of biocompatible material tubing (which exhibits super elastic or shape memory properties, e.g. Nitinol). This feature dramatically reduces the possibility of an unintentional separation of the treatment device from the delivery wire.

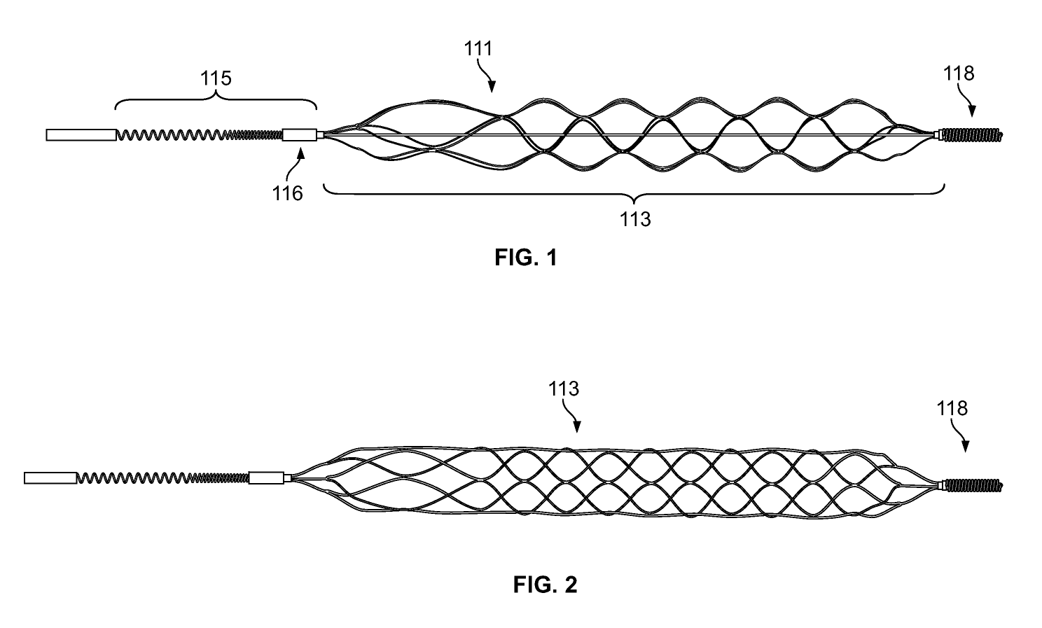

Turning now to the drawings, FIG. 1 and FIG. 2 each shows an example of the overall profile of device 111. Device 111 can be made from one piece of Nitinol super elastic material or Nitinol shape memory alloy tubing. It is also made from other biocompatible materials that exhibit super elastic or shape memory properties. The device is made by laser cutting, mechanical machining, chemical machining, electrochemical machining, EDM, and related techniques known to artisans.

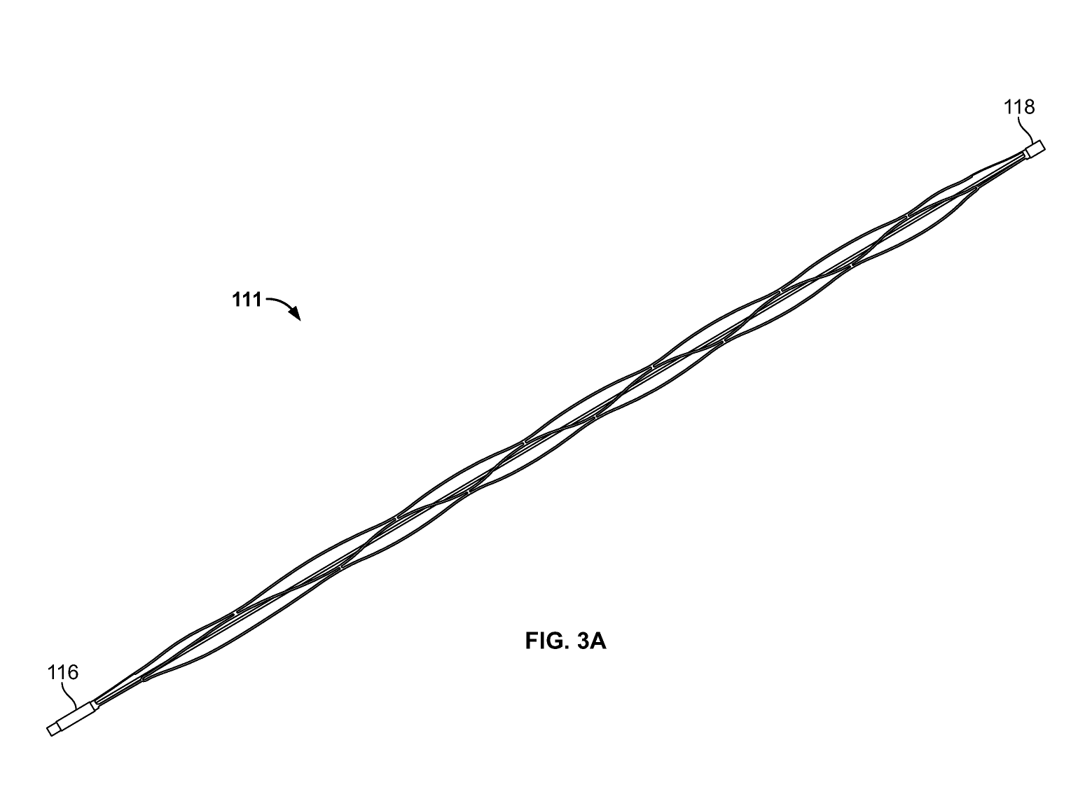

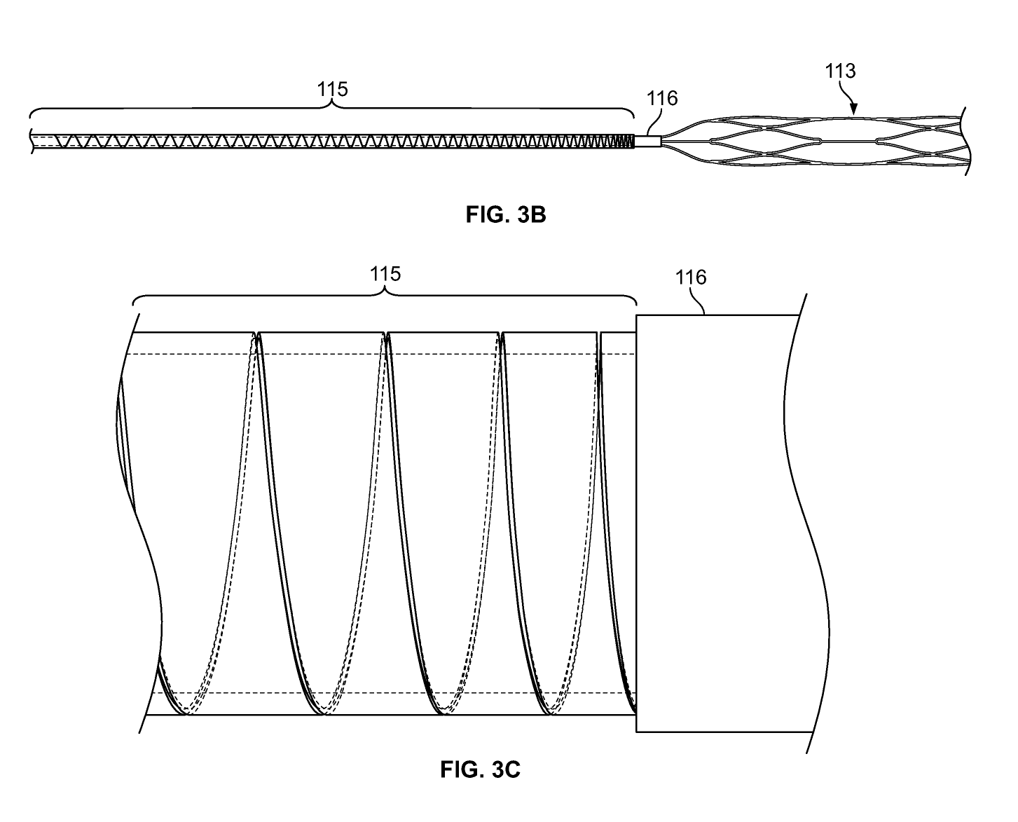

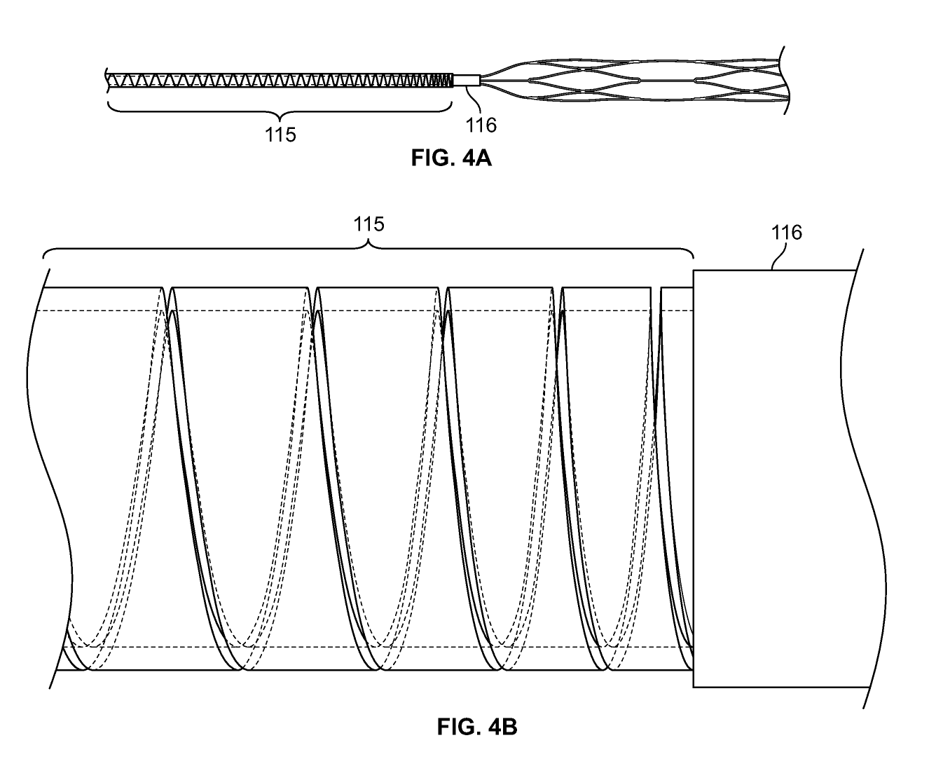

Treatment portion 113 is bordered on either end by proximal marker 116 and distal marker 118. Transition portion 115 is further detailed in FIGS. 3 and 4.

FIG. 2 shows details of an embodiment with novel structures in treatment portion 113.

FIGS. 3A through 3C show examples of the transition portion 115 of the design. Transition portion can be 3A.) a straight piece of tubing; 3B.) a tubing with spiral cut through the entire wall thickness; 3C.) a tubing with spiral cut partially through the entire wall thickness. Other geometries can also be cut with an unlimited number of variations.

FIGS. 4A and 4B show examples of the transition portion 115 with spiral configurations. The pitch size can vary along the length for varying flexibilities. The spiral cut can either be through the entire wall thickness of the tubing or only partially through the wall thickness leaving a “groove” on the surface. In the case the spiral cut is through the entire wall thickness, the transition portion will have a real spiral profile (FIG. 4B).

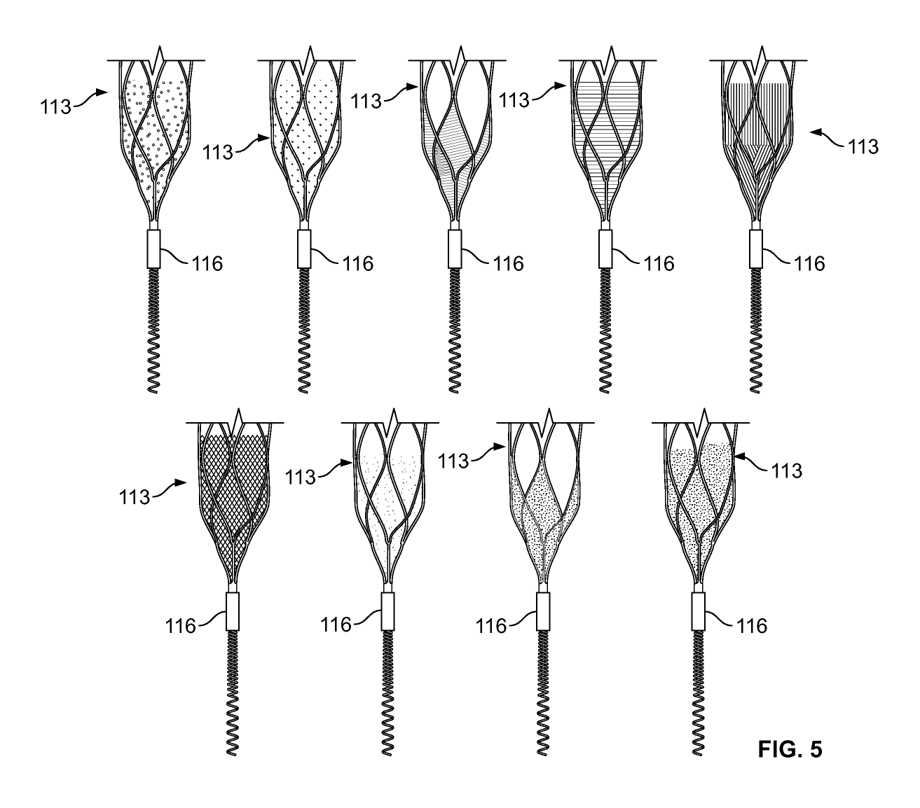

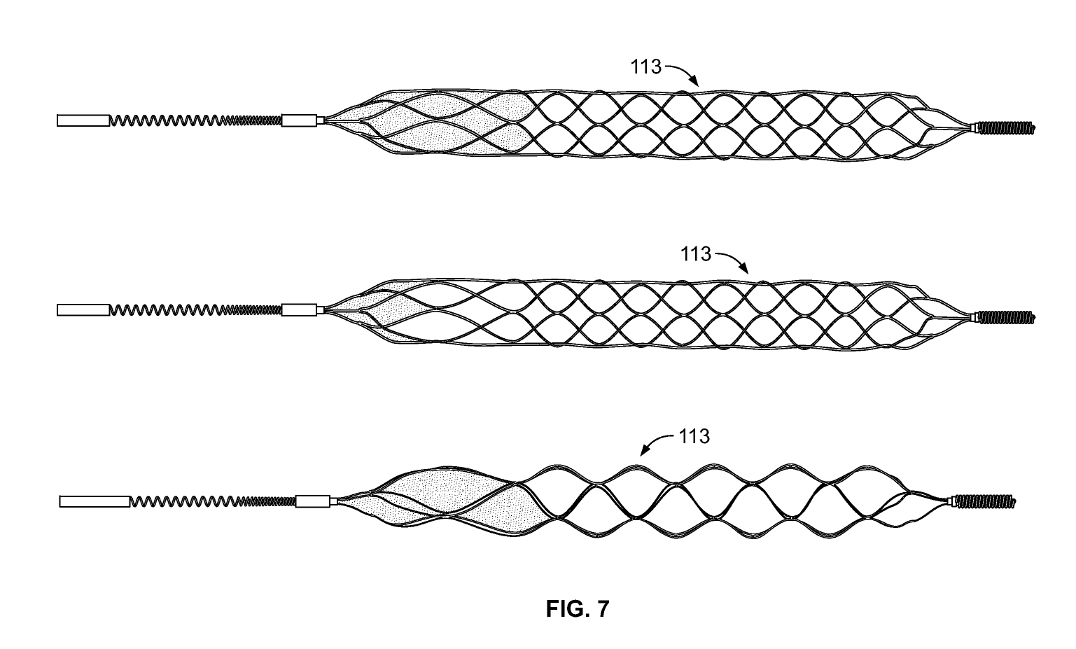

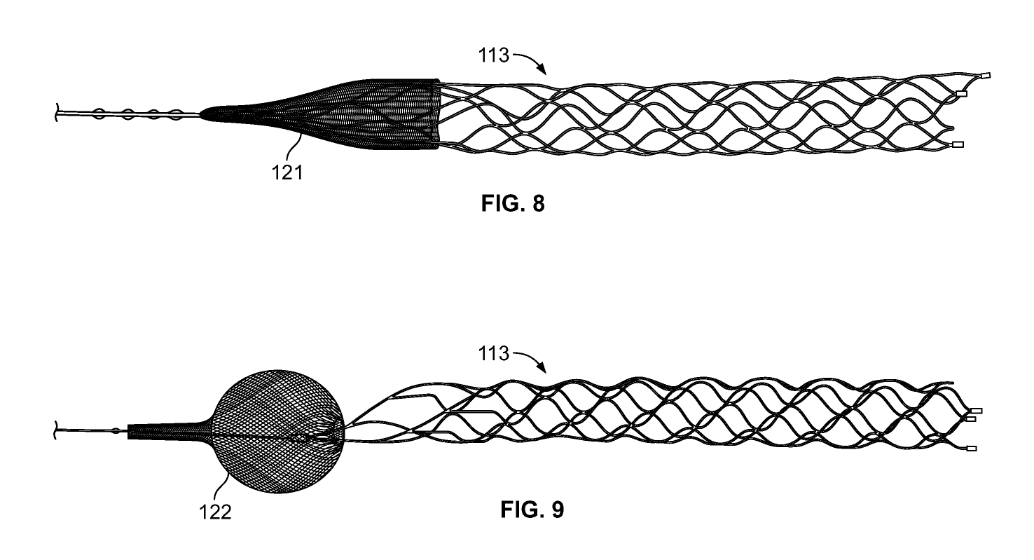

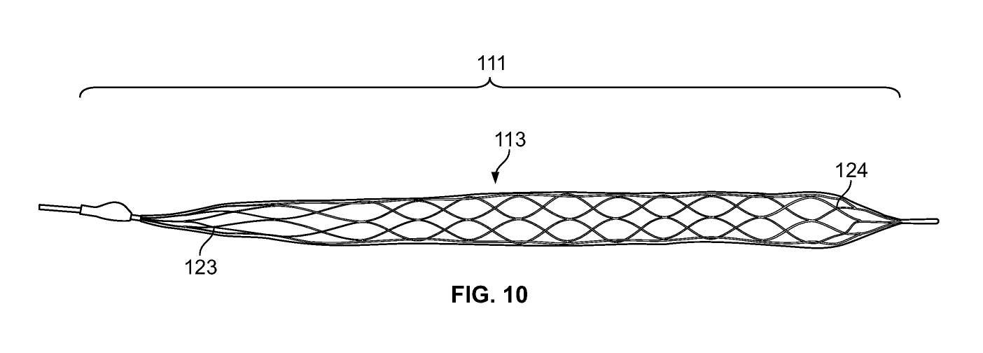

FIGS. 5 and 7 show the exemplary configurations of proximal flow block feature/element 113 on the proximal portion of the expandable portion.

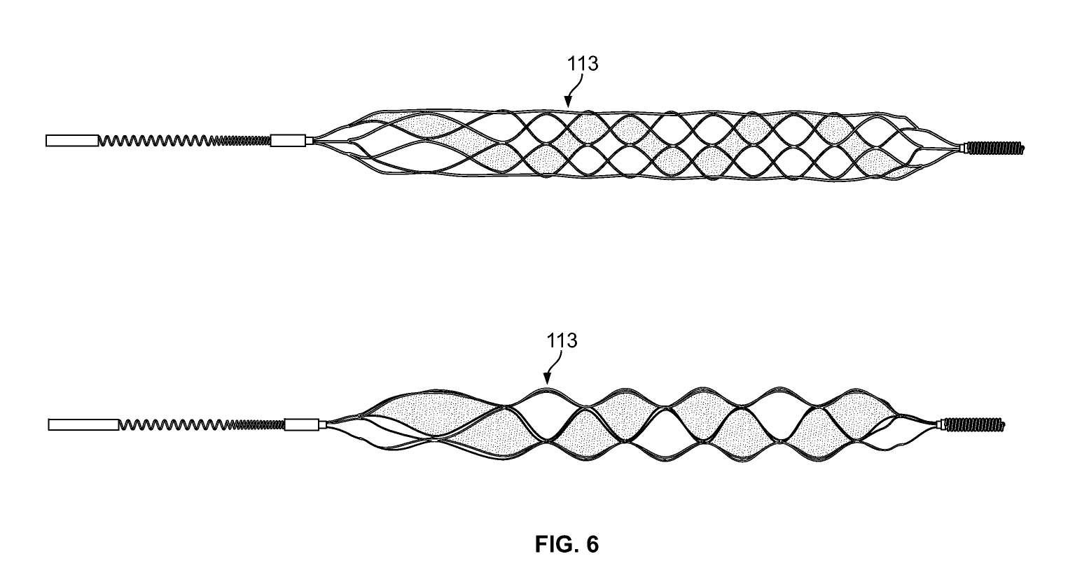

FIG. 6 shows the exemplary configurations of the flow block feature/element on the treatment portion 113 of the expandable portion.

FIG. 8 shows an exemplary configuration of the proximal flow block/restriction feature/element 113 with braided wire tubular structure 121 from metallic or polymer materials.

Artisans readily understand that the proximal flow block/restriction structure can be part or away from the proximal body of the device. The proximal flow block/restriction structure can have a first smaller compacted profile to make the delivery through microcatheter possible. The proximal flow block/restriction structure can have a second larger expanded diameter/profile when the device is released from the microcatheter or other delivery system to block, limit, or restrict the blood flow.

FIG. 9 shows an exemplary configuration of the proximal flow block/restriction feature/element 113 with spherical or near spherical structure from metallic or polymer materials 122. The spherical structure can be braided or laser cut structure. It can be fabricated from the one or two element(s) of the device or fabricated from other pieces of material, then is attached onto the device proximal end by mechanical means, or thermal (laser or soldering) process, or adhesive/glue, or heat shrink technology.

The proximal flow block/restriction structure can be part or away from the proximal body of the device. The proximal flow block/restriction structure can have a first smaller compacted profile to make the delivery through microcatheter possible. The proximal flow block/restriction structure can have a second larger expanded diameter/profile when the device is released from the microcatheter or other delivery system to block or limit, restrict the blood flow. One example is that the spherical or near spherical structure is made from braided metallic or polymer wires, then is attached onto the proximal portion of the device. One end (either proximal or distal end) of the spherical structure 122 can be loose or free to move, to accommodate the length change or variation during the delivery and expansion processes. The spherical structure can also be fabricated from the same piece of Nitinol tubing with that of the device by laser cutting or chemical processes and then shape set to a larger diameter than the raw Nitinol tubing.

FIG. 10 shows an exemplary configuration of a clot removal device 111 with flow block feature 113 and “wells” 123/125 in the cell space. The flow block feature described here can be made from polymer materials, the polymer metal can block the lumen of the device and also form “wells” or volume at each cell space to house the clot and prevent the clot to be break off or loose during the procedure.

The proximal portion design can be a straight tubing portion. The proximal flow block/restriction feature can be combined and used with any existing mechanical clot retriever to help remove the clot from vasculature.

Radiopaque markers can be attached on any portion of the device for positioning. One way to gain the full visibility for the device is to run a radiopaque material through the entire or partial lumen of the delivery wire. Markers can also be placed on the treatment portion to aid in positioning.

The device can have surface treatment on various portions to improve performance for the various portions of the device. The proximal and transition portion can either be coated or covered by typical biocompatible materials for lubricity entirely or partially. The surface of the distal treatment portion can have either a positive or negative charge for improved clot adhesion. The surface of the distal treatment portion can also be either mechanically or chemically treated to have a “rough” surface for improved clot adhesion. The “rough” surface can be achieved by 1.) Porous surface coating or layer; 2.) Micro blasted surface or micropinning; 3.) Irregular strut geometry or arrangement.

It will be appreciated by those skilled in the art that changes could be made to the example embodiments described in this invention without departing from the broad invention concept/idea thereof. While particular embodiments of the present invention have been described, it is not intended to limit the invention only to any specific embodiment.

While methods, devices, compositions, and the like, have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims. It is understood that the term, present disclosure, in the context of a description of a component, characteristic, or step, of one particular embodiment of the disclosure, does not imply or mean that all embodiments of the disclosure comprise that particular component, characteristic, or step.

It should also be understood that a variety of changes may be made without departing from the essence of the disclosure. Such changes are also implicitly included in the description. They still fall within the scope of this disclosure. It should be understood that this disclosure is intended to yield a patent covering numerous aspects of the disclosure both independently and as an overall system and in both method and apparatus modes.

Further, each of the various elements of the disclosure and claims may also be achieved in a variety of manners. This disclosure should be understood to encompass each such variation, be it a variation of an implementation of any apparatus implementation, a method or process implementation, or even merely a variation of any element of these.

Particularly, it should be understood that as the disclosure relates to elements of the disclosure, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this disclosure is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these disclosure(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular implementation, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative implementations.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps. Such terms should be interpreted in their most expansive forms so as to afford the applicant the broadest coverage legally permissible.

1. A system and device with proximal flow block feature/elements, comprising, in combination:
a proximal block feature being cell structures with smaller cell spaces, polymer cover, fabric/textures, polymer net, and/or net made from biocompatible metallic materials.

2. The system and device of claim 1, wherein the proximal block feature can block blood flow through the device and treatment vessel segment when the device is deployed during use; and,
wherein the proximal block feature can restrict blood flow through the device and treatment vessel segment when the device is deployed during use.

3. The system and device of claim 2, wherein the block feature can be integrated into the proximal portion or element of the device to block the lumen when the device is deployed during use.

4. The system and device of claim 2, wherein the block feature can be a part or away, have distance from the proximal portion or element of the device to block the lumen when the device is deployed during use.

5. The system and device of claim 2, wherein the proximal block portion can block blood flow during the application, and to eliminate or reduce the risk to break the clots or flush clots to distal.

6. The system and device of claim 2, wherein the proximal block portion can reduce blood flow during the application, and to eliminate or reduce the risk to break the clots or flush clots to distal.

7. The system and device of claim 2, wherein the proximal block portion will contact with vessel wall when the device is expanded and block blood flow during use.

8. The system and device of claim 1, wherein the proximal block portion length can be in the range from 1 to 90% of the total device treatment portion length.

9. The system and device of claim 1, wherein the proximal block portion length can be in the range from 1 to 80% of the total device treatment portion length.

10. The system and device of claim 1, wherein the proximal block portion length can be in the range from 1 to 70% of the total device treatment portion length.

11. The system and device of claim 1, wherein the proximal block portion length can be in the range from 1 to 60% of the total device treatment portion length.

12. The system and device of claim 1, wherein the proximal block portion length can be in the range from 1 to 50% of the total device treatment portion length.

13. The system and device of claim 1, wherein the proximal block portion length can be in the range from 1 to 40% of the total device treatment portion length.

14. The system and device of claim 1, wherein the proximal block portion length can be in the range from 1 to 30% of the total device treatment portion length.

15. The system and device of claim 1, wherein the proximal block portion length can be in the range from at least about 1 to approximately 20% of the total device treatment portion length.

16. A mechanical thrombectomy device with proximal flow restriction feature/elements, further comprising:
the proximal restriction feature can be cell structure with smaller cell space, can be polymer cover, can be fabric/textures, can be polymer net, can be cover and/or net made from biocompatible metallic materials;
the proximal restriction feature can block blood flow through the device and treatment vessel segment when the device is deployed during use;
the proximal restriction feature can restrict blood flow through the device and treatment vessel segment when the device is deployed during use;
the proximal restriction feature can limit blood flow through the device and treatment vessel segment when the device is deployed during use;
the restriction feature can be integrated into the proximal portion or element of the device to block the lumen when the device is deployed during use;
the restriction feature can be a part or away, have distance from the proximal portion or element of the device to block the lumen when the device is deployed during use;
the proximal restriction portion can block blood flow during the application, and to eliminate or reduce the risk to break the clots or flush clots to distal;
the proximal restriction portion can reduce blood flow during the application, and to eliminate or reduce the risk to break the clots or flush clots to distal; and,
The proximal restriction portion will contact with vessel wall when the device is expanded and block blood flow during use.

17. A mechanical thrombectomy device with flow block feature/elements in the body portion of the expandable portion, wherein
the block feature can be integrated into the body portion of the device along the radials or length directions; and further wherein;
the projected cell structure along the device lumen, polymer cover on the “peaks” or “valleys” along the length, fabric/textures on the “peaks” or “valleys” along the length, polymer net on the “peaks” or “valleys” along the length, cover and/or net made from biocompatible metallic materials on the “peaks” or “valleys” along the length, PVD, or CVD, or laser, or plasma deposited thin films.

18. A mechanical thrombectomy device system, further comprising:
having a delivery portion and treatment portion fabricated from a single piece of Nitinol super elastic material or Nitinol shape memory alloy tubing;
made from other biocompatible materials which exhibit super-elastic or shape memory properties;
made by laser cutting, mechanical machining, chemical machining, electro chemical machining, or EDM, wherein;
a distal portion is the treatment portion and can be fabricated from the same piece of tubing with the delivery portion; and,
desired diameter and length can be achieved through heat setting process and/or mechanical forming method.

19. The mechanical thrombectomy device of claim 10, the transition portion comprising:)
1.) a straight piece of tubing;
2.) a tubing with spiral cut through the entire wall thickness;
3.) a tubing with spiral cut not through the entire wall thickness;
4.) other geometry variations as known to artisans.

20. The mechanical thrombectomy device of claim 19, wherein:
the proximal portion can be straight tubing;
the proximal and transition portion can either be coated or covered by typical biocompatible materials for lubricity entirely or partially; and,
the surface of the distal treatment portion can have either positive or negative charge for improved clot adhesion;
the surface of the distal treatment portion can also be either mechanically or chemically treated to have a “rough” surface for improved clot adhesion;
the “rough” surface can be achieved by 1.) porous surface coating or layer; 2.) microblasted surface or micropinning; 3.) irregular strut geometry or arrangement;
there is a spiral configuration for the device strut arrangement along the length direction of the device;
the spiral “groove” forms the “volume” in the device, and helps for “housing” the clot to prevent or reduce the possibility that the clot is break off or loose during the procedure.
